(19) United States
(12) Patent Application Publication
(10) Pub. No.: US 2021/0017484 A1
(43) Pub. Date: Jan. 21, 2021

(54) CELL STRAINER

(71) Applicant: ENPLAS CORPORATION, Kawaguchi-shi, Saitama (JP)

(72) Inventor: Kenjiro TAKI, Saitama (JP)

(73) Assignee: Enplas Corporation, Kawaguchi-shi, Saitama (JP)

(21) Appl. No.: 16/320,347

(22) PCT Filed: Jun. 13, 2017
(86) PCT No.: PCT/JP2017/021754

§ 371 Date: Jan. 24, 2019

(30) Foreign Application Priority Data
Jul. 27, 2016 (JP) 2016-147130

(51) Int. Cl. C12M 1/26

(52) U.S. Cl. CPC C12M 33/14

(57) ABSTRACT

A cell strainer causes fluid to flow out in a tube via a filter or causes fluid in the tube to flow in via the filter by an operation of a pipette engaged with the tube. The filter is formed into a shape such that the filter is entirely positioned in the tube when the cell strainer is engaged with the tube. A tip engaging hole press-fitted to a distal end of a tip for pipette is formed in the cell strainer. A flow passage communicating with the tip engaging hole is formed inside the cell strainer. The filter has a plurality of openings having similar shapes. The openings communicate between an inside of the flow passage and an internal space of the tube to filter out a substance larger than the openings among substances in the fluid.

### TECHNICAL FIELD

This invention relates to a cell strainer used to screen aggregated cells into single cells and obtain uniform single cell suspension.

### BACKGROUND ART

As illustrated in FIG. 10, a cell strainer 100 conventionally known generally has a tubular body with a closed bottom. The cell strainer 100 includes a flange portion 101 on an upper opening end, a grip portion 102 extending outward from this flange portion 101, and a filter portion 103 constituting a part of a cylindrical surface extending downward from the flange portion 101. The filter portion 103 of this cell strainer 100 is inserted into a tube 104 for use with the flange portion 101 mounted on an upper opening edge 105 of the tube 104. As the filter portion 103, a mesh filter made of nylon of 40 to 200 μm (micrometer) is used. The tube 104 of 50 ml (milliliter) is used (see Patent Document 1).

A work using the cell strainer 100 illustrated in FIG. 10 produces, for example, suspension of various organs and cultured cells in a test tube and the like, scoops up the suspension with a pipette (not illustrated) and a tip 106 for pipette, injects the suspension into the cell strainer 100 mounted on the upper opening edge 105 of the tube 104 from a distal end of the tip 106, and removes impurities such as bone chips and cell supporting tissue in the suspension with the filter portion 103 to obtain the single cell suspension in the tube 104.
Patent Document 1: JP-UM-B-7-2007

### DISCLOSURE OF THE INVENTION

### Problems to be Solved by the Invention

However, the conventional cell strainer 100 is used mounted on the upper opening edge 105 of the tube 104 and a clearance between the distal end of the tip 106 and an upper opening 107 of the cell strainer 100 is large. Accordingly, there has been a possibility that the suspension discharged from the tip 106 scatters outside from the clearance between the tip 106 and the upper opening 107 and this causes contamination or contaminates the surrounding work environment.

Therefore, the present invention provides a cell strainer configured to prevent the contamination and the contamination of an external environment.

### Solutions to the Problems

The present invention relates to a cell strainer 1 that causes fluid to flow out in a tube 3 via a filter 4 or causes fluid in the tube 3 to flow in via the filter 4 by an operation of a pipette engaged with the tube 3. With the cell strainer 1 according to the present invention, the filter 4 is formed into a shape such that the filter 4 is entirely positioned in the tube 3 when the cell strainer 1 is engaged with the tube 3. With the cell strainer 1 according to the present invention, a tip engaging hole 28 press-fitted to a distal end of a tip 2 mounted to the pipette is formed. A flow passage 36 communicating with the tip engaging hole 28 is internally formed. With the cell strainer 1 according to the present invention, the filter 4 has a plurality of openings 18 having similar shapes. The openings 18 communicate between an inside of the flow passage 36 and an internal space of the tube 3 to filter out a substance larger than the openings 18 among substances in the fluid.

### Effects of the Invention

The cell strainer according to the present invention is formed into the shape such that filter is entirely positioned in the tube during a work. Moreover, since the tip is press-fitted to the tip engaging hole in the cell strainer according to the present invention, the fluid discharged from the tip into the flow passage does not scatter outside the tube or does not leak to the outside of the tube. Moreover, the fluid flown into the flow passage from the inside of the tube via the filter does not scatter outside the tube or does not leak to the outside of the tube. Consequently, the cell strainer according to embodiments does not cause contamination and does not contaminate a surrounding work environment in a work that screens aggregated cells into single cells and obtains uniform single cell suspension.

### BRIEF DESCRIPTION OF THE DRAWINGS

FIG. 1 is a drawing illustrating a use state of a cell strainer according to a first embodiment of the present invention.

FIG. 2 includes drawings illustrating the cell strainer according to the first embodiment of the present invention. FIG. 2A is a plan view of the cell strainer. FIG. 2B is a side view of the cell strainer. FIG. 2C is a cross-sectional view of the cell strainer illustrated taken along the line A1-A1 of FIG. 2A. FIG. 2D is a cross-sectional view of the cell strainer illustrated taken along the line A2-A2 of FIG. 2A. FIG. 2E is a cross-sectional view of the cell strainer illustrated taken along the line A3-A3 of FIG. 2C. FIG. 2F is a bottom view of the cell strainer when viewed in the arrow B1 direction of FIG. 2B.

FIG. 3 includes drawings illustrating an outer frame body of the cell strainer according to the first embodiment. FIG. 3A is a plan view of the outer frame body. FIG. 3B is a side view of the outer frame body. FIG. 3C is a bottom view of the outer frame body when viewed in the arrow B2 direction of FIG. 3B. FIG. 3D is a cross-sectional view of the outer frame body illustrated taken along the line A4-A4 of FIG. 3A. FIG. 3E is an enlarged view of the arrow C1 part. FIG. 3F is an enlarged view of the arrow C2 part. FIG. 3G is an enlarged view of the arrow C3 part. FIG. 3H is an enlarged view of the arrow C4 part.

FIG. 4 includes drawings illustrating an upper structure of the cell strainer according to the first embodiment. FIG. 4A is a plan view of the upper structure. FIG. 4B is a side view of the upper structure. FIG. 4C is a cross-sectional view of the upper structure illustrated taken along the line A5-A5 of FIG. 4A. FIG. 4D is a bottom view of the upper structure.

FIG. 5 includes drawings illustrating an intermediate structure of the cell strainer according to the first embodiment. FIG. 5A is a plan view of the intermediate structure. FIG. 5B is a cross-sectional view of the intermediate structure illustrated taken along the line A6-A6 of FIG. 5A. FIG. 5C is a side view of the intermediate structure.

FIG. 6 includes drawings illustrating a lower structure of the cell strainer according to the first embodiment. FIG. 6A is a plan view of the lower structure. FIG. 6B is a side view of the lower structure when viewed in the arrow B3 direction of FIG. 6A. FIG. 6C is a side view of the lower structure when viewed in the arrow B4 direction.

FIG. 7 is a drawing illustrating a use state of a cell strainer according to a second embodiment of the present invention.

FIG. 8 includes drawings illustrating the cell strainer according to the second embodiment of the present invention. FIG. 8A is a plan view of the cell strainer. FIG. 8B is a side view of the cell strainer. FIG. 8C is a cross-sectional view of the cell strainer illustrated taken along the line A7-A7 of FIG. 8A. FIG. 8D is a cross-sectional view of the cell strainer illustrated taken along the line A8-A8 of FIG. 8A.

FIG. 9 includes drawings illustrating an upper structure of the cell strainer according to the second embodiment. FIG. 9A is a plan view of the upper structure. FIG. 9B is a side view of the upper structure. FIG. 9C is a cross-sectional view of the upper structure illustrated taken along the line A9-A9 of FIG. 9A. FIG. 9D is a cross-sectional view of the upper structure illustrated taken along the line A10-A10 of FIG. 9A. FIG. 9E is a bottom view of the upper structure.

FIG. 10 is a drawing describing a use state of a conventional cell strainer.

### DESCRIPTION OF PREFERRED EMBODIMENTS

Embodiments of the present invention are described in detail with reference to drawings hereinafter.

### First Embodiment

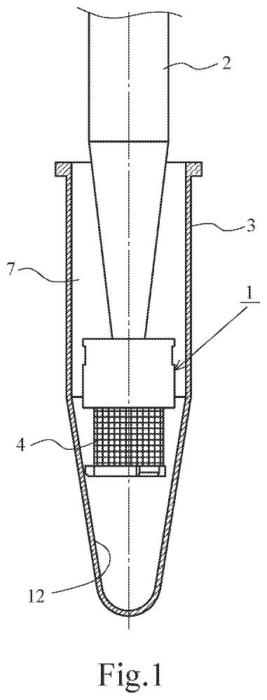

FIG. 1 is a drawing illustrating a use state of a cell strainer 1 according to the embodiment. As illustrated in FIG. 1, the cell strainer 1 according to the embodiment is engaged with a tube 3 while press-fitted to a distal end of a tip 2 mounted to a pipette (not illustrated), and the cell strainer 1 is entirely housed inside the tube 3 for use.

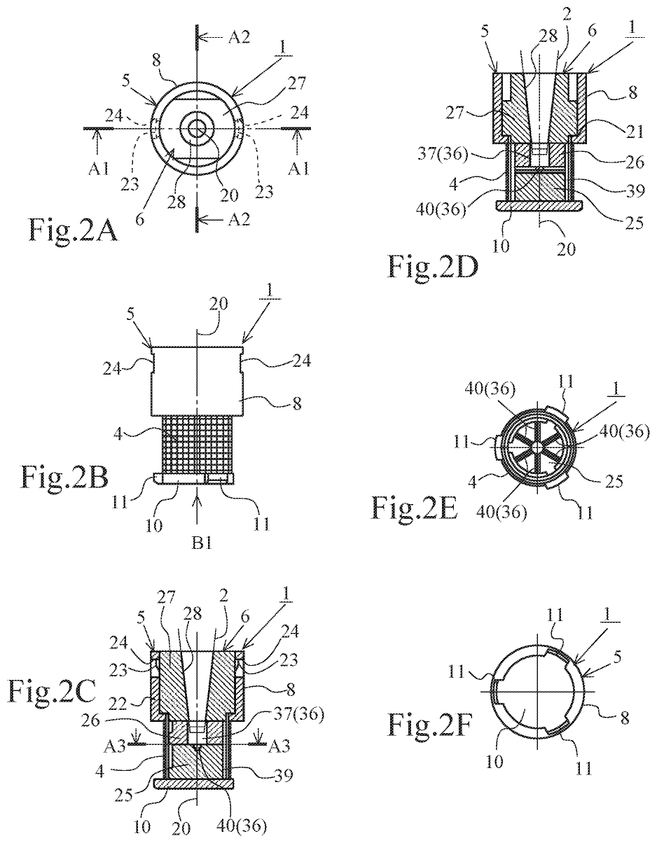

FIG. 2 includes drawings illustrating the cell strainer 1 according to the embodiment. FIG. 2A is a plan view of the cell strainer 1. FIG. 2B is a side view of the cell strainer 1. FIG. 2C is a cross-sectional view of the cell strainer 1 illustrated taken along the line A1-A1 of FIG. 2A. FIG. 2D is a cross-sectional view of the cell strainer 1 illustrated taken along the line A2-A2 of FIG. 2A. FIG. 2E is a cross-sectional view of the cell strainer 1 illustrated taken along the line A3-A3 of FIG. 2C. FIG. 2F is a bottom view of the cell strainer 1 when viewed in the arrow B1 direction of FIG. 2B.

As illustrated in FIG. 2, the cell strainer 1 includes an outer frame body 5 and an internal structure 6. The outer frame body 5 has a tubular shape with a closed bottom with which a filter 4 is integrally formed. The internal structure 6 is housed in this outer frame body 5. This cell strainer 1 is formed into a shape such that the cell strainer 1 is entirely housed in an internal space 7 of the tube 3 (see FIG. 1).

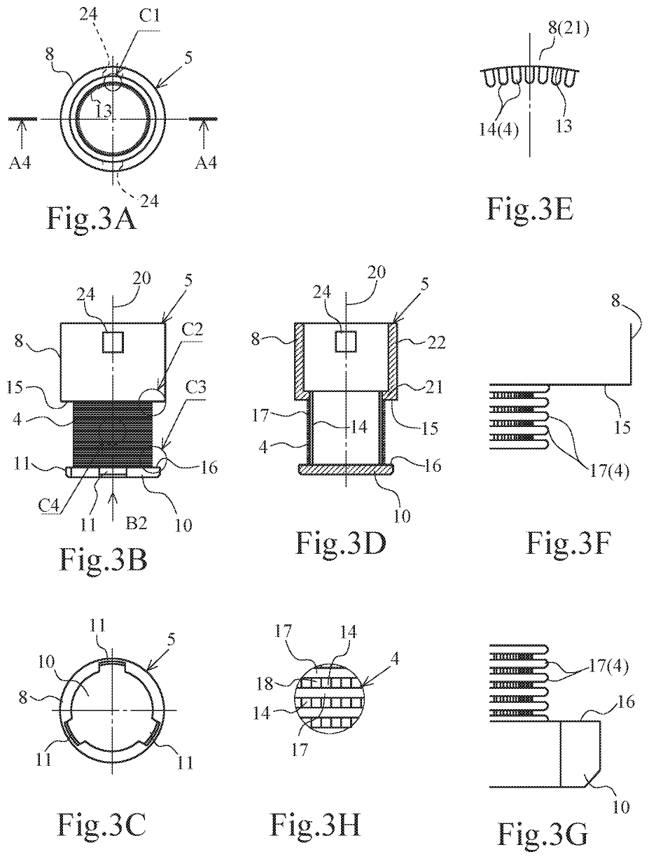

As illustrated in FIG. 2 and FIG. 3, the outer frame body 5 includes a cylindrical upper frame body 8, the cylindrical filter 4, and a circular plate-shaped bottom plate 10. The filter 4 is integrally formed with the lower surface of this upper frame body 8 and is concentrically formed with the upper frame body 8. The bottom plate 10 is integrally formed with the lower end of this filter 4. The outer frame body 5 is integrally formed (injection-molded) with a synthetic resin material (for example, polypropylene (PP), polyethylene (PE), and polyamide (PA)).

As illustrated in FIG. 1 to FIG. 3, the bottom plate 10 of the outer frame body 5 has an outer peripheral surface on which positioning protrusions 11 are formed at three locations at regular intervals. The positioning protrusions 11 abut on a tapered inner surface 12 on the distal end side of the tube 3 to position the cell strainer 1 in the tube 3. By bringing the positioning protrusions 11 into abutment with the tapered inner surface 12 of the tube 3, this bottom plate 10 secures a constant clearance between the filter 4 and the inner surface of the tube 3.

As illustrated in FIG. 1 to FIG. 3, the filter 4 of the outer frame body 5 includes a plurality of regular quadrilateral openings 18 at regular intervals formed of a plurality of longitudinal ribs 14 and a plurality of ring-shaped cross ribs 17. The longitudinal ribs 14 are formed at regular intervals along a peripheral edge of a bottom hole 13 of the upper frame body 8. The cross ribs 17 are formed at regular intervals between a lower surface 15 of the upper frame body 8 and an upper surface 16 of the bottom plate 10. In this filter 4, the longitudinal ribs 14 positioned on the radially inward side are formed along a center axis 20, the cross ribs 17 positioned on the radially outward side are formed into the ring shapes so as to be perpendicular to the longitudinal ribs 14, and the longitudinal ribs 14 and the cross ribs 17 are integrated at intersecting portions. One side of the opening 18 is formed to have a length of, for example, 0.04 mm, 0.07 mm, or 0.1 mm. An optimum value according to a size of a substance (for example, a cell) needs to be filtered out is determined as the value of the length.

As illustrated in FIG. 2 and FIG. 3, the upper frame body 8 of the outer frame body 5 includes a bottom surface part 21 and a cylindrical wall part 22. The bottom surface part 21 projects radially outward from the upper end portion of the filter 4. The cylindrical wall part 22 extends upward from an end radially outward of this bottom surface part 21. On this bottom surface part 21 of the upper frame body 8, the bottom hole 13 where the longitudinal ribs 14 of the filter 4 are formed is formed. On the cylindrical wall part 22 of the upper frame body 8, a pair of lock holes 24, 24 engaged with a pair of lock claws 23, 23 are formed opposed to one another. The pair of lock claws 23, 23 described later are formed on an upper structure 27 of the internal structure 6.

As illustrated in FIG. 2, the internal structure 6 includes a lower structure 25, an intermediate structure 26, and the upper structure 27. The lower structure 25 is housed in the internal space of the filter 4 and on the bottom plate 10. The intermediate structure 26 is stacked on the upper surface of the lower structure 25 in the internal space of the filter 4. The upper structure 27 is housed in the internal space of the upper frame body 8 and is stacked on the upper surface of the intermediate structure 26. The internal structure 6 is housed in the internal space of the outer frame body 5 in the order from the lower structure 25, the intermediate structure 26, and the upper structure 27.

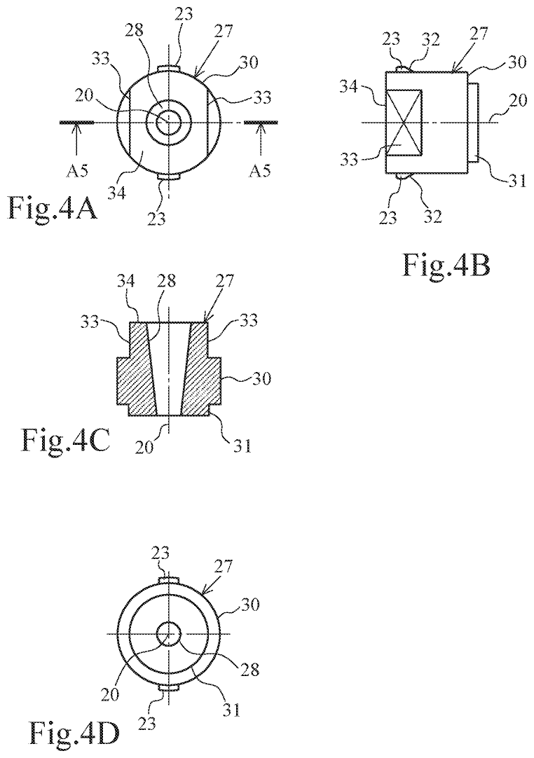

As illustrated in FIG. 2 and FIG. 4, the upper structure 27 has the tapered tip engaging hole 28 where the taped distal end side of the tip 2 is press-fitted along the center axis 20. At least an upper end edge or a lower end edge of this tip engaging hole 28 closely contacts the tapered distal end side of the tip 2, and the tip engaging hole 28 penetrates from the upper surface to the lower surface of the upper structure 27. The upper structure 27 further includes a large-diameter portion 30 engaged with the cylindrical wall part 22 of the upper frame body 8 and a small-diameter portion 31 engaged with the radially inward side of the longitudinal ribs 14 of the filter 4. Additionally, the upper structure 27 includes the pair of lock claws 23, 23 on the upper portion of the outer peripheral surface. This lock claw 23 has an inclined surface 32 extending obliquely upward from the outer peripheral surface of the upper structure 27 so as to ensure smooth engagement with the lock hole 24 while elastically deforming the periphery of the lock hole 24 of the upper frame body 8 when the upper structure 27 is engaged with the upper frame body 8. When the lock claws 23 on the upper structure 27 are engaged with the lock holes 24, the peripheral parts of the lock holes 24 of the upper frame body 8 elastically restore the original shapes, and the lock claws 23 are engaged into the lock holes 24, thus retaining the upper structure 27. For easy of grasp from the above with an assembling tool or a similar tool, a width-across-flat part 34 having a pair of parallel surfaces 33, 33 is formed on this upper structure 27.

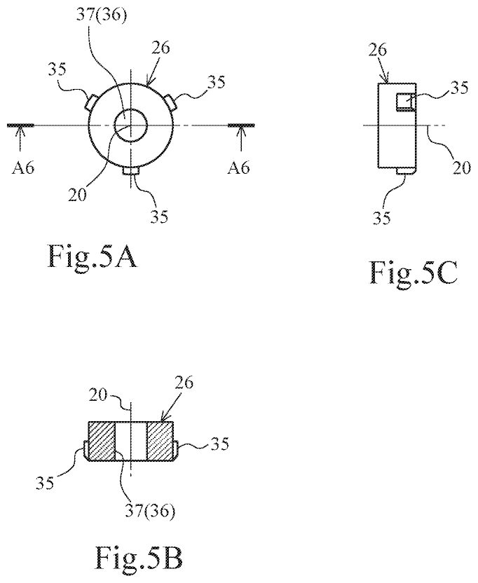

As illustrated in FIG. 2 and FIG. 5, the intermediate structure 26 is formed into a columnar shape and includes protrusions 35 at three locations at regular intervals on the outer peripheral surface. These protrusions 35 on the intermediate structure 26 have functions to secure an approximately cylindrical clearance 39 between the outer peripheral surface of the intermediate structure 26 and the filter 4 and align the center axis 20 of the upper structure 27 and the center axis 20 of the intermediate structure 26. The intermediate structure 26 includes a first flow passage 37 (flow passage 36) communicating with the tip engaging hole 28 of the upper structure 27 along the center axis 20. This first flow passage 37 is a circular hole that penetrates the intermediate structure 26 along the center axis 20, opens to the upper surface and the lower surface of the intermediate structure 26, and has a hole diameter slightly larger than a hole diameter (a hole diameter as the smallest diameter part of the tapered tip engaging hole 28) of the lower end edge of the tip engaging hole 28. Even when manufacturing errors and similar errors of the outer frame body 5, the upper structure 27, and the intermediate structure 26 are accumulated, the first flow passage 37 allows entering the distal end of the tip 2 inserted into the tip engaging hole 28 to the inside without a collision with the upper surface of the intermediate structure 26. Consequently, the tip 2 is surely press-fitted to the tip engaging hole 28 of the upper structure 27 and therefore the tip engaging hole 28 can be sealed. Accordingly, fluid discharged from the distal end of the tip 2 does not leak to the upper surface side of the upper structure 27 via the tip engaging hole 28.

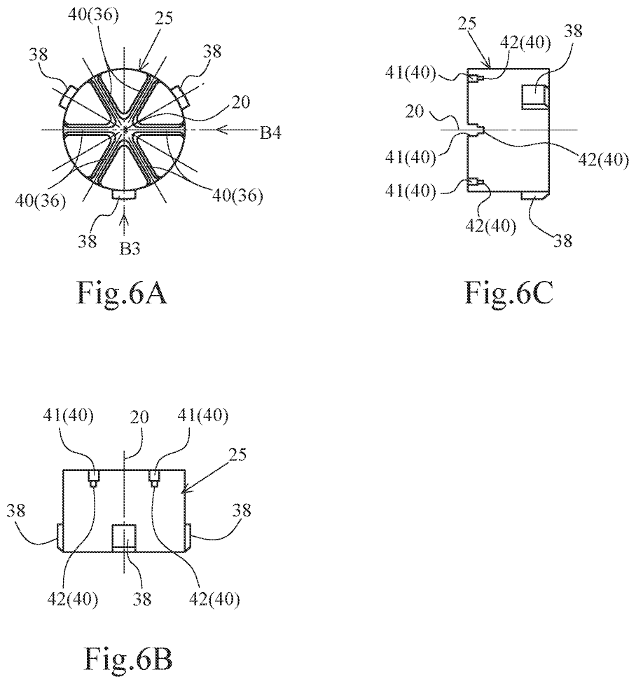

As illustrated in FIG. 2 and FIG. 6, the lower structure 25 is formed into a columnar shape with an outer diameter dimension identical to that of the intermediate structure 26 and includes protrusions 38 at three locations at regular intervals on the outer peripheral surface. Similarly to the protrusions 35 on the intermediate structure 26, these protrusions 38 on the lower structure 25 have functions to secure the approximately cylindrical clearance 39 between the outer peripheral surface of the lower structure 25 and the filter 4 and align the center axis 20 of the intermediate structure 26 and the center axis 20 of the lower structure 25. Additionally, the lower structure 25 includes second flow passages 40 on the upper surface opposed to the intermediate structure 26. The second flow passages 40 communicate with the first flow passage 37 of the intermediate structure 26 to constitute the flow passage 36 together with the first flow passage 37. The second flow passages 40 are radial grooves formed in a radial pattern at six locations at regular intervals radially outward from the center of the lower structure 25. The first flow passage 37 of the intermediate structure 26 opens at the joining part of these radial grooves at the six locations. This second flow passage 40 includes a first radial groove part 41 opening to the upper surface of the lower structure 25 and a second radial groove part 42 opening to the bottom surface of this first radial groove part 41. The second radial groove part 42 has a groove width narrower than that of the first radial groove part 41. Thus, the radial grooves at the six locations constituting the second flow passages 40 have the two-stage groove structure, the first radial groove parts 41 and the second radial groove parts 42, ensuring obtaining an effect that a clogging due to the substance in the fluid is less likely to occur. Additionally, in the second flow passages 40, the adjacent radial grooves (41, 42) are rounded off into arc shapes on the joining sides, and the end sides radially outward of the radial grooves (41, 42) are formed so as to smoothly expand the flow passage widths into the arc shapes, thus ensuring reducing pressure loss of the fluid.

As illustrated in FIG. 1, the cell strainer 1 according to the embodiment configured as described above is press-fitted to the distal end of the tip 2 mounted to the pipette (not illustrated) and is inserted into the tube 3 together with the tip 2 until the positioning protrusions 11 abut on the tapered inner surface 12 of the tube 3. In the cell strainer 1, after the positioning protrusions 11 abut on the tapered inner surface 12 of the tube 3 to be positioned, when the pipette is operated and the fluid is discharged from the tip 2 into the flow passage 36 (first flow passage 37), the fluid is flown out to the filter 4 via the first flow passage 37 and the second flow passages 40. In this respect, the cell strainer 1 guides the fluid up to a position opposed to the filter 4 with the flow passage 36 (the first flow passage 37 and the second flow passages 40), which is formed at the inside (the intermediate structure 26 and the lower structure 25), without releasing pressure of the fluid discharged from the tip 2 to the atmosphere and causes the fluid to flow out from the end radially outward of the flow passage 36 (second flow passages 40) to the filter 4. The cell strainer 1 is configured such that the fluid flows out from the end radially outward of the flow passage 36 (second flow passages 40) to the filter 4 passes through the filter 4 and then flows out into the tube 3, or after flowing through the clearance 39 between the outer peripheral surface of the intermediate structure 26 and the outer peripheral surface of the lower structure 25; and the filter 4, the fluid passes through the filter 4 and flows out into the tube 3. When the fluid passes through the filter 4, this cell strainer 1 filters out substances larger than the openings 18 of the filter 4. Consequently, the cell strainer 1 of the embodiment is configured to screen aggregated cells into single cells and also configured to obtain (configured to accumulate) uniform single cell suspension in the tube 3.

As described above, as illustrated in FIG. 1, during the work, the cell strainer 1 according to the embodiment is press-fitted to the distal end of the tip 2, which is mounted to the pipette (not illustrated) and is inserted into the tube 3 together with the tip 2 until the positioning protrusions 11 abut on the tapered inner surface 12 of the tube 3. Thus, the cell strainer 1 according to the embodiment is formed into the shape configured to be housed in the tube 3 during the work. Moreover, since the tip 2 is press-fitted to the tip engaging hole 28 in the cell strainer 1 according to the embodiment, the fluid discharged from the tip 2 into the first flow passage 37 (into the flow passage 36) of the intermediate structure 26 does not scatter outside the tube 3 or does not leak to the outside of the tube 3. Consequently, the cell strainer 1 according to the embodiment does not cause contamination and does not contaminate the surrounding work environment in the work that screens the aggregated cells into the single cells and obtains the uniform single cell suspension.

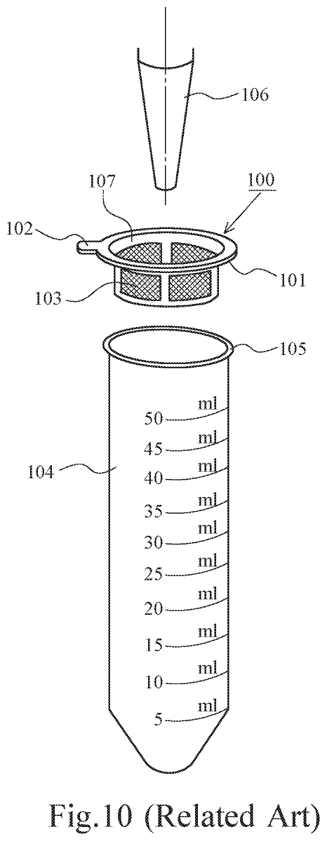

The cell strainer 1 according to the embodiment is configured such that the pressure of the fluid discharged from the tip 2 to the flow passage 36 of the internal structure 6 is not released to the atmosphere up to the position opposed to the filter 4 and the interval between the end radially outward of the flow passage 36 (second flow passages 40) and the filter 4 is held constant. Therefore, the pipette easily applies the pressure to the fluid heading from the end radially outward of the flow passage 36 to the filter 4. Consequently, compared with the conventional example, the cell strainer 1 according to the embodiment facilitates performing the work that screens the aggregated cells into the single cells and the work that obtains the uniform single cell suspension. Compared with the cell strainer 1 according to the embodiment, a conventional cell strainer 100 illustrated in FIG. 10 has a large clearance with a tip 106 mounted to a pipette and therefore pressure of fluid (suspension) discharged from the tip 106 lowers greatly. Therefore, a technique has been required for the work that eliminates the aggregation of the cells and screens the cells into the single cells.

Additionally, the cell strainer 1 according to the embodiment can uniformly flow out the fluid from the second flow passages 40 (flow passage 36), which are radially positioned at the six locations, to the cylindrical filter 4; therefore, the fluid passes through the wide area of the filter 4. Accordingly, the clogging is less likely to occur and the working efficiency can be improved.

Further, since the cell strainer 1 according to the embodiment includes the openings 18 of the filter 4 highly accurately shaped by injection molding, the clogging is less likely to occur and a variation of the sizes of the substances to be filtered out in the fluid is small (a high accurate filter function is provided) compared with a sintering cell strainer (not illustrated) where variations of sizes and shapes of openings are large.

The filter 4 of the cell strainer 1 according to the embodiment is highly accurately shaped by injection molding and is less likely to deform. Therefore, compared with a filter (not illustrated) produced by weaving nylon fiber, the filter 4 features the small variation of the sizes of the substances to be filtered out in the fluid (the high accurate filter function is provided).

Since the cell strainer 1 according to the embodiment is press-fitted to the distal end of the tip 2 for use, the cell strainer 1 can be configured to have the size fitting the used pipette and the tube 3 can be downsized.

With the cell strainer 1 according to the embodiment, the lower structure 25, the intermediate structure 26, and the upper structure 27 individually molded by injection molding may be integrated by ultrasonic welding and the like. These lower structure 25, intermediate structure 26, and upper structure 27 are made of, for example, a synthetic resin material (for example, polypropylene (PP), polyethylene (PE), and polyamide (PA)).

### Second Embodiment

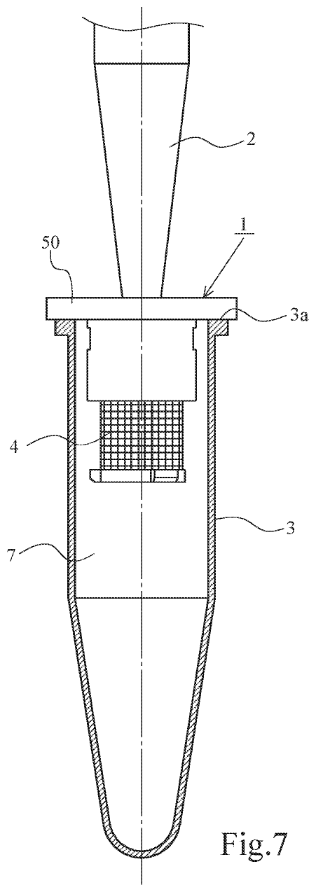

FIG. 7 is a drawing illustrating a use state of the cell strainer 1 according to the embodiment. As illustrated in FIG. 7, the cell strainer 1 according to the embodiment is press-fitted to the distal end of the tip 2 mounted to the pipette for use. The cell strainer 1 includes a flange portion 50 on the upper end mounted on an opening end 3a of the tube 3 and other portions (such as the filter 4) excluding the flange portion 50 are positioned inside the tube 3 for use.

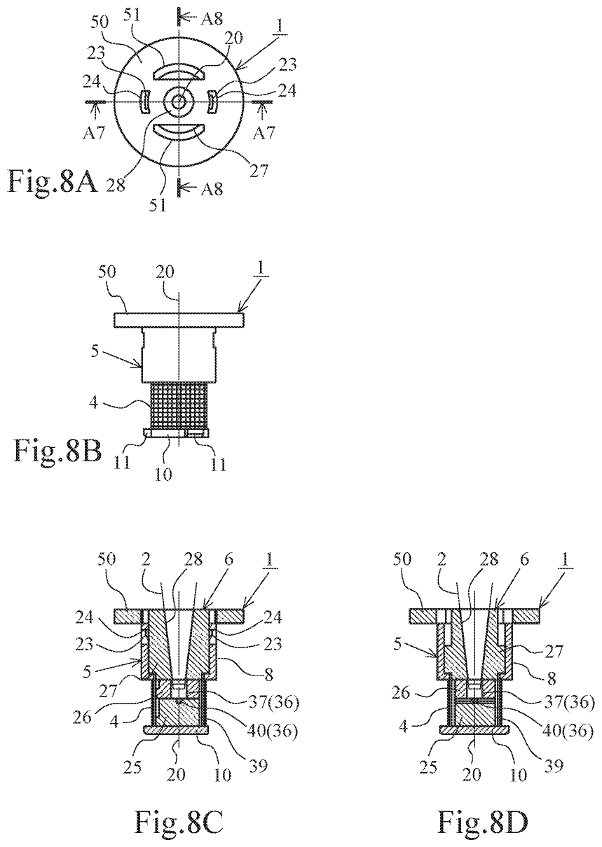

FIG. 8 includes drawings illustrating the cell strainer 1 according to the embodiment. FIG. 8A is a plan view of the cell strainer 1. FIG. 8B is a side view of the cell strainer 1. FIG. 8C is a cross-sectional view of the cell strainer 1 illustrated taken along the line A7-A7 of FIG. 8A. FIG. 8D is a cross-sectional view of the cell strainer 1 illustrated taken along the line A8-A8 of FIG. 8A.

The cell strainer 1 according to the embodiment illustrated in FIG. 8 is similar to the cell strainer 1 according to the first embodiment excluding the point that the flange portion 50 mounted on the opening end 3a of the tube 3 is formed on the upper portion of the upper structure 27. Accordingly, the identical reference numerals are attached to the parts of the cell strainer 1 according to the embodiment common in the cell strainer 1 according to the first embodiment, and the overlapping description will be omitted.

In the cell strainer 1 according to the embodiment, the flange portion 50 formed on the upper end portion of the upper structure 27 is positioned so as to cover the upper end surface of the upper frame body 8 of the outer frame body 5.

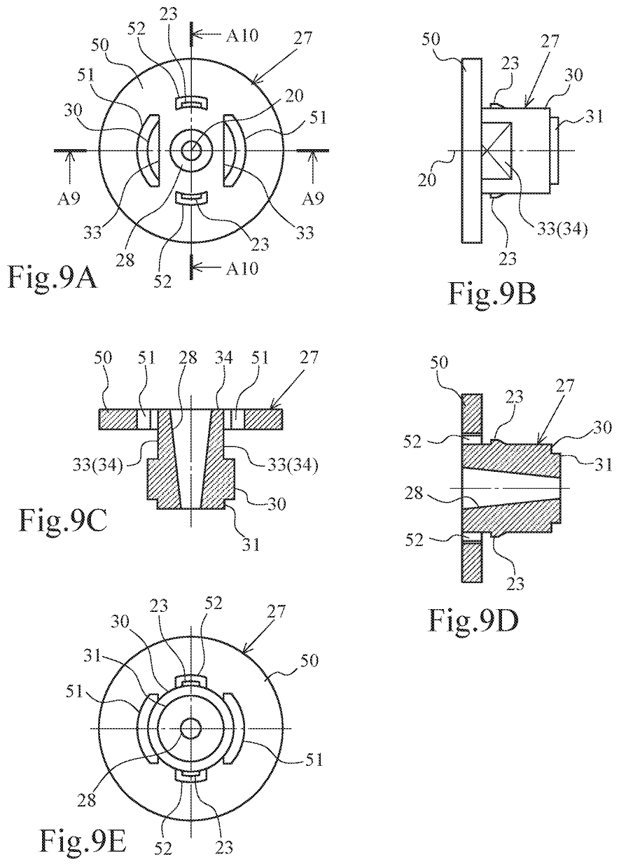

As details are illustrated in FIG. 9, the upper structure 27 has a shape where the flange portion 50 is integrally formed with the upper end portion of the upper structure 27 of the cell strainer 1 according to the first embodiment (see FIG. 4). This upper structure 27 includes the tip engaging hole 28 at the center of the flange portion 50 and includes a pair of first mold release holes 51, 51 opposed such that between which this tip engaging hole 28 is interposed. These first mold release holes 51, 51 are release holes for a mold to shape the width-across-flat part 34 having the pair of parallel surfaces 33, 33 and allow the molding of the width-across-flat part 34 serving as an undercut on the flange portion 50. The upper structure 27 has a pair of second mold release holes 52, 52 at positions displaced by 90° with respect to the first mold release holes 51, 51. These second mold release holes 52, 52 are release holes for the mold to shape the lock claw 23, 23 and allow the molding of the lock claw 23, 23 serving as undercuts on the flange portion 50.

The cell strainer 1 according to the embodiment is configured to have a shape such that when the tip 2 is press-fitted to the tip engaging hole 28 of the upper structure 27 and the flange portion 50 is mounted on the opening end 3a of the tube 3 for use, the other parts (such as the filter 4) other than the flange portion 50 are housed in the tube 3 (see FIG. 7). Accordingly, the cell strainer 1 according to the embodiment can obtain the effect similar to that of the cell strainer 1 according to the first embodiment where the cell strainer 1 is entirely housed in the tube 3.

### Other Embodiments

The cell strainer 1 according to the present invention is not limited to the above-described respective embodiments and may be used such that the pipette suctions the fluid in the tube 3 into the tip 2 via the filter 4 and the flow passage 36. The cell strainer 1 of the present invention thus configured can filter out substances having sizes equal to or more than the sizes of the openings 18 of the filter 4 and obtain (suck) the fluid containing substances less than the sizes of the openings 18 in the tip 2.

The cell strainer 1 according to the present invention is not limited to the above-described respective embodiments and the opening 18 of the filter 4 may have a shape other than the regular quadrilateral such as a circular shape and a hexagonal shape (multangular shape).

The outer frame body 5 of the cell strainer 1 according to the present invention is not limited to the case where the outer frame body 5 is entirely produced by injection molding and may be produced by insert molding using a filter formed by weaving nylon fiber into a grid pattern in advance. With the outer frame body 5 thus produced by insert molding using the filter formed by weaving the nylon fiber in the grid pattern in advance, the fibers of the filter are possibly displaced and openings have shapes similar to the shapes (square shapes other than the regular quadrilateral) of the openings 18 of the filter 4 produced by injection molding. Accordingly, when the high accurate filter function is required for the cell strainer 1, the use of the outer frame body 5 having the openings 18 of the filter 4 all highly accurately injection-molded into the identical shape, the regular quadrilateral, is preferred.

While the embodiments of the cell strainer 1 according to the present invention where the filter 4 is formed into the cylindrical shape have been described, this should not be construed in a limiting sense. The filter 4 may be modified into any shape including a tapered shape according to the use state.

### DESCRIPTION OF REFERENCE SIGNS

1: Cell strainer
2: Tip
3: Tube
4: Filter
18: Opening
28: Tip engaging hole
36: Flow passage

1. A cell strainer that causes fluid to flow out in a tube via a filter or causes fluid in the tube to flow in via the filter by an operation of a pipette engaged with the tube, wherein
the filter is formed into a shape such that the filter is entirely positioned in the tube when the cell strainer is engaged with the tube,
a tip engaging hole press-fitted to a distal end of a tip mounted to the pipette is formed, and a flow passage communicating with the tip engaging hole is internally formed, and
the filter has a plurality of openings having similar shapes, and the openings communicate between an inside of the flow passage and an internal space of the tube to filter out a substance larger than the openings among substances in the fluid.

2. The cell strainer according to claim 1, comprising:
a tubular outer frame body with a closed bottom integrally including the filter; and
an internal structure housed in the outer frame body, wherein
the tip engaging hole is formed along an axial center of the internal structure,
the flow passage includes a first flow passage and a plurality of second flow passages, the first flow passage is formed along the axial center of the internal structure, and the plurality of second flow passages are branched from the first flow passage to the filter, and
the fluid injected from the tip into the flow passage in a pressurized state is guided to end portions of the second flow passages opposed to the filter without being released to an atmosphere.

3. The cell strainer according to claim 2, wherein
the filter is formed on the outer frame body so as to be opposed to an inner peripheral surface of the tube with the cell strainer mounted to the tube, and the filter is formed to be positioned away from the inner peripheral surface of the tube.

4. The cell strainer according to claim 2, wherein
the outer frame body is integrally molded by injection molding.

5. The cell strainer according to claim 1, wherein
the openings have rectangular shapes in plan view, and the openings are formed so as to have sizes identical to those of the other adjacent openings.

6. The cell strainer according to claim 3, wherein
the outer frame body is integrally molded by injection molding.

7. The cell strainer according to claim 2, wherein
the openings have rectangular shapes in plan view, and the openings are formed so as to have sizes identical to those of the other adjacent openings.

8. The cell strainer according to claim 3, wherein
the openings have rectangular shapes in plan view, and the openings are formed so as to have sizes identical to those of the other adjacent openings.

9. The cell strainer according to claim 4, wherein
the openings have rectangular shapes in plan view, and the openings are formed so as to have sizes identical to those of the other adjacent openings.

10. The cell strainer according to claim 6, wherein
the openings have rectangular shapes in plan view, and the openings are formed so as to have sizes identical to those of the other adjacent openings.
